(19) United States
(12) Patent Application Publication
(10) Pub. No.: US 2026/0091174 A1
(43) Pub. Date: Apr. 2, 2026

(54) SPACER FOR THE CONTROLLED ADMINISTRATION OF AN ACTIVE INGREDIENT

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/343,410

(22) Filed: Sep. 29, 2025

(30) Foreign Application Priority Data
Oct. 2, 2024 (EP) 24204112.7

(51) Int. Cl. A61M 5/168; A61F 2/48; A61F 2/30

(52) U.S. Cl. CPC A61M 5/16881; A61F 2/484; A61F 2002/3068; A61M 2205/0216; A61M 2205/04

(57) ABSTRACT

An implantable spacer for delivering a medical fluid to a patient, wherein the spacer has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves.

### CROSS-REFERENCE TO RELATED APPLICATIONS

This application claims priority pursuant to 35 U.S.C. 119(a) to European Application No. 24204112.7 filed October 2, 2024, which application is incorporated herein by reference in its entirety.

### FIELD OF THE INVENTION

The present invention relates to a spacer for repeated or continuous local release of active ingredients. Furthermore, procedures for local release of active ingredients are described.

### BACKGROUND

The term “spacer” usually refers to orthopedic implants that can be implanted in a patient as a temporary placeholder, usually after previous debridement of the infected tissue. Spacers can be modeled after the shape of hip and knee joints or other joints or, in particular for use in infected long bones, can have the shape of intramedullary nails. Another application of spacers is to calm infections through the local release of antimicrobial active ingredients in previously debrided bone and soft tissue. Conventional spacers are usually made of PMMA bone cement and contain one or more antibiotics embedded in the spacer material. These antimicrobial active ingredients are released from the PMMA bone cement after implantation by exposure to aqueous body fluids such as wound secretions and blood. The release of active ingredients occurs through diffusion processes and leads to an initial high release and a subsequent release of small amounts of active ingredient. However, it would be more desirable to have a temporally constant release of sufficiently high amounts of active ingredient to ensure consistent local concentrations of active ingredient over a period of several days to weeks.

EP3763335B1 describes a knee joint for the release of drugs, which has a supply line for active ingredient solutions and in which the active ingredient solution can reach a plurality of outlet openings via a channel system. The problem here is that the openings arranged directly in the vicinity of the supply line release larger volumes of liquid, while the more distant outlet openings can only release small volumes or no volumes of liquid. Furthermore, coagulated blood or ingrown connective tissue can clog the outlet openings.

A similar knee joint spacer system was disclosed in U.S. Patent No. 10,864,314.

In WO2016205077A, spacers with a rinsing function were described, the rinsing liquid being guided on the spacer surface through grooves on the spacer surface.

EP3542759B1 proposes a similar spacer system which has outlet openings and also openings for draining the rinsing liquid.

Intramedullary nails, intended to calm infections, were also disclosed. These intramedullary nails have a wall perforated with openings. Antibiotic or antiseptic solutions can be introduced into the intramedullary nails from outside the patient through a connected tube (U.S. Patent No. 5,681,289, CN2857862Y, CN201370624Y, WO2016205077A1). The antibiotic or antiseptic solutions exit through openings in the intramedullary nail wall. The problem with these concepts is that the openings can become clogged by coagulated blood and that the openings can be partially or completely closed after a few days by ingrown connective tissue. A further problem is that antibiotic or antiseptic solutions tend to leak from the openings of the intramedullary nail that are located directly next to the tube connector. Only small volumes of the antibiotic or antiseptic solutions are released through openings further away from the introductory tube, because the solution exits the intramedullary nails via the shortest route behind the introductory tube. The release of constant volumes of the antibiotic or antiseptic solutions over the entire length of the intramedullary nails is therefore not guaranteed.

### SUMMARY

An object of the present invention is to solve one or more of the problems described above and further problems of the prior art.

The present invention is based in particular on the object of providing a spacer which enables repeated or continuous release of active ingredients on the outer surface of the spacer over several days to weeks. For this purpose, a medical fluid, such as aqueous active ingredient solutions, can be introduced into the spacer from the outside via a supply line, for example, and the fluid can then exit from a plurality of delivery valves on the outer surface of the spacer. The spacer according to the present invention is preferably designed so that approximately equal volumes of the active ingredient solution can be released simultaneously from all delivery valves. Furthermore, clogging of the delivery valves by coagulated blood and ingrown connective tissue can preferably be avoided. It is also desirable that liquids from outside cannot penetrate through the delivery valves into the interior of the spacer. The delivery valves may be designed so that they do not protrude beyond the outer surface of the spacer and so that no components of the spacer are forced into the surrounding tissue during delivery of medical fluid. The spacer can enable the delivery of relatively small volumes of a medical fluid, for example in the range of up to a maximum of 50 mL per single application. The spacer may also be suitable for delivering medical fluid with a high concentration of active ingredient. The spacer according to the present invention is preferably suitable for the precisely controlled release of active ingredients, allowing uniform release to take place at different locations on the spacer. This is an advantage over conventional spacer systems, which aim to provide a rinsing function by introducing larger volumes into the spacer to rinse the surrounding tissue. According to the present invention, the spacer described herein preferably does not require any further elements for draining or reabsorbing a rinsing liquid. The spacer may be suitable for temporary implantation into previously infected and debrided bone cavities.

These objects are achieved by the methods, devices, kits and medical uses described herein, in particular those which are described in the claims.

Preferred embodiments of the present invention will be described below.

A first embodiment of a first aspect of the present invention relates to an implantable spacer for delivering a medical fluid to a patient, wherein the spacer has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves.

A second embodiment of the present invention relates to a spacer according to the first embodiment, wherein the delivery valves each have a sleeve-shaped housing, and wherein the spacer has a plurality of receptacles, wherein the delivery valves can be connected to the spacer by positive and/or non-positive engagement of the housings into the receptacles.

A third embodiment of the present invention relates to a spacer according to any of the preceding embodiments, wherein the delivery valves each comprise a first material, wherein the first material is rubber-elastic, and the first material further comprises a slit.

A fourth embodiment of the present invention relates to an implantable spacer according to the third embodiment, wherein the slits are configured to reversibly open and close by means of a resilient restoring force of the first material.

A fifth embodiment of the present invention relates to an implantable spacer according to the third or fourth embodiment, wherein the first material has a Shore A hardness of 30 to 80, preferably 40 to 70, more preferably 50 to 60.

A sixth embodiment of the present invention relates to an implantable spacer according to any of the preceding embodiments, wherein the channel has an inlet opening for receiving a fluid, wherein the inlet opening is preferably arranged on the outer surface of the spacer.

A seventh embodiment of the present invention relates to an implantable spacer according to the second and sixth embodiment, wherein the inlet opening is designed in an identical manner to the receptacles, or wherein the inlet opening is designed in a manner different from the receptacles.

An eighth embodiment of the present invention relates to an implantable spacer according to any of the preceding embodiments, wherein the spacer is selected from the group consisting of a knee joint spacer, a hip joint spacer, a vertebral body spacer and an intramedullary nail spacer.

A ninth embodiment of the present invention relates to an implantable spacer according to any of the preceding embodiments, wherein the spacer is designed and configured to deliver a substantially identical amount of fluid from each of the delivery valves simultaneously.

A tenth embodiment of the present invention relates to an implantable spacer according to any of the preceding embodiments, wherein the spacer is designed and configured to open and/or close all delivery valves simultaneously.

An eleventh embodiment of the present invention relates to an implantable spacer according to any of the preceding embodiments, wherein the delivery valves are each arranged such that they are flush with the outer surface of the spacer.

A twelfth embodiment of the present invention relates to an implantable spacer according to any of the preceding embodiments, wherein the channel has a plurality of branches, and wherein preferably each branch has a delivery valve or an inlet opening according to the sixth embodiment.

A thirteenth embodiment relates to an implantable spacer according to any of the preceding embodiments, wherein the delivery valves are designed and configured to allow liquid to pass only unidirectionally through the delivery valves.

A second aspect of the present invention relates to a medical fluid for use in a medical procedure, wherein the procedure comprises the following steps:

providing an implantable spacer according to any of the preceding embodiments,

introducing a medical fluid into the channel of the spacer,

pressurizing the medical fluid in the channel to open the delivery valves in a pressure-dependent manner and deliver the fluid to a patient.

A further embodiment according to the second aspect of the present invention relates to a fluid for use according to the preceding embodiment, wherein the fluid comprises an active ingredient selected from the group consisting of an antibiotic, an antiphlogistic agent, an anesthetic agent and a cytostatic agent.

### BRIEF DESCRIPTION OF THE DRAWINGS

FIG. 1 shows a spacer according to the present invention, which is designed as a hip joint spacer;

FIG. 2 shows a spacer according to the present invention which is filled with a medical fluid;

FIG. 3 shows an embodiment of a delivery valve;

FIG. 4 shows a section of a spacer according to the present invention with a modular delivery valve;

FIG. 5 shows a delivery valve in a fully open state;

FIG. 6 shows a delivery valve in a partially open state;

FIG. 7 shows a delivery valve in a closed state;

FIG. 8 shows a spacer according to the present invention, which is designed as a knee joint spacer;

FIG. 9 shows a knee joint spacer according to the present invention with joint elements;

FIG. 10 shows a spacer according to the present invention, which is designed as a vertebral body spacer; and,

FIG. 11 shows a spacer according to the present invention, which is designed as an intramedullary nail spacer.

### DETAILED DESCRIPTION OF THE INVENTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” ”containing,” “have,” or “having.”

“Operatively connected” or “operatively connectable” means herein that two elements appertaining thereto have a functional relationship to one another. For example, a first element may be configured to control or move a second element through such an operative connection. The term “control” here also comprises blocking or enabling a function, for example allowing or restricting the movement or other function of an element.

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, all the features described herein in connection with a method are in principle also considered to be applicable to the products, devices, kits and uses described herein, and vice versa. All such considered combinations are not explicitly listed in all instances, simply to keep the description brief. Technical solutions known to be equivalent to the features described herein are also intended in principle to be encompassed by the scope of the present invention.

The technical norms and standards described herein, for example in conjunction with test procedures, refer to the version current on the priority date of the present application.

One aspect of the present invention relates to an implantable spacer for delivering a medical fluid to a patient, wherein the spacer has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves.

The term “spacer” refers herein to a medical implant which is designed and configured to be implanted in a patient as a temporary placeholder in place of a bone or joint, or a part thereof. In one embodiment, the spacer comprises a biocompatible material such as PMMA, stainless steel or titanium. The spacer may comprise or consist of a metal, a plastics material or a metal-plastics composite. Examples of biocompatible metals include 316L steel, cobalt-chromium steel, titanium, and titanium alloys. Examples of biocompatible plastics materials are polymethyl methacrylate, polyamide 12, polyethersulfone and polyetherketone. In one embodiment, the spacer consists of at least 90% (mass/mass) PMMA.

In one embodiment, the spacer according to the present invention can be produced or is produced by SLM (selective laser melting) or by EBM (electron beam melting) from stainless steel or titanium or other biocompatible metals or alloys. In one embodiment, the spacer according to the present invention can be produced or is produced by SLS (selective laser sintering) from suitable plastics materials, such as polyamide 12 or polymethyl methacrylate. In one embodiment, the spacer according to the present invention can be produced or is produced by plastics injection molding from thermoplastics. In this case, the spacer can be assembled from a plurality of injection-molded parts, it being possible for these parts to be connected to one another, for example, by welding or gluing.

The spacer described herein is preferably designed and configured to deliver a medical fluid. A “medical fluid” refers herein to a fluid which is intended for medical use and has a medicinal effect.

The term “medical fluid” herein refers in particular to aqueous and non-aqueous liquids which may contain dissolved active ingredients, in particular pharmaceutical active ingredients, or which may themselves have a medicinal effect. This term also includes gases and gas-liquid mixtures that can exert a pharmacological effect in the human or animal organism. In one embodiment, the medical fluid comprises an active ingredient. In one embodiment, the active ingredient is selected from the group consisting of an antibiotic, an antifungal agent, a cytostatic agent, an anesthetic, an osteoinductive active ingredient, and antiphlogistic agent.

In one embodiment, the active ingredient is an antibiotic. In one embodiment, the antibiotic is selected from the group consisting of penicillins, cephalosporins, carbapenems, quinolones, macrolides, lincosamides, aminoglycosides and glycopeptides. Examples of penicillins are amoxicillin and benzylpenicillin. Examples of cephalosporins are ceftriaxone and cefuroxime. Examples of carbapenems are meropenem and imipenem. Examples of quinolones are ciprofloxacin and levofloxacin. Examples of macrolides are azithromycin and clarithromycin. Examples of glycopeptides are vancomycin and teicoplanin. Examples of aminoglycosides are gentamicin and tobramycin. An example of an aminoglycoside is clindamycin.

In one embodiment, the active ingredient is an antimycotic. Examples of antimycotics comprise polyenes (e.g., amphotericin B, nystatin, natamycin), azoles (e.g., fluconazole, voriconazole), echinocandins (e.g., caspofungin, micafungin), and allylamines (e.g., terbinafine).

In one embodiment, the active ingredient is a cytostatic agent. Examples of cytostatic agents comprise alkylating substances, antimetabolites, natural products, protein kinase inhibitors and monoclonal antibodies. Examples of alkylating substances comprise cyclophosphamide, melphalan and busulfan. Examples of antimetabolites comprise methotrexate, 5-fluorouracil, and gemcitabine. Examples of natural products comprise paclitaxel, doxorubicin and vincristine. Examples of protein kinase inhibitors comprise imatinib, gefitinib, and sunitinib. Examples of monoclonal antibodies comprise rituximab, trastuzumab and bevacizumab.

In one embodiment, the active ingredient is an anesthetic. Examples of anesthetics include lidocaine, bupivacaine, ropivacaine, propofol, etomidate, ketamine, morphine, fentanyl, and remifentanyl.

In a further embodiment, the active ingredient is an osteoinductive active ingredient. Examples of osteoinductive active ingredients comprise bone morphogenetic proteins (BMPs), parathyroid hormone-related peptides, anti-sclerostin antibodies, and growth factors. Examples of bone morphogenetic proteins comprise BMP-2 and BMP-7. An example of a parathyroid hormone-related peptide is teriparatide (PTH 1-34). An example of an anti-sclerostin antibody is romosozumab. Examples of growth factors comprise fibroblast growth factors (FGFs) and platelet derived growth factor (PDGF).

In a further embodiment, the active ingredient is an antiphlogistic agent. Examples of antiphlogistic agents comprise nonsteroidal anti-inflammatory drugs (NSAIDs), glucocorticoids, selective COX-2 inhibitors, biologics (e.g., TNF-α inhibitors), and Janus kinase inhibitors. Examples of nonsteroidal anti-inflammatory drugs (NSAIDs) comprise ibuprofen, diclofenac, and naproxen. Examples of glucocorticoids comprise prednisone, dexamethasone, and hydrocortisone. Examples of selective COX-2 inhibitors comprise celecoxib and etoricoxib. Examples of TNF-α inhibitors comprise infliximab, adalimumab, and etanercept. Examples of Janus kinase inhibitors comprise tofacitinib and baricitinib.

In one embodiment, the active ingredient is suitable for treating a bone disease. In one embodiment, the active ingredient is selected from the group of bisphosphonates (e.g., alendronate, zoledronate), calcitonin, selective estrogen receptor modulators (e.g., raloxifene) and strontium ranelate. In one embodiment, the active ingredient comprises hyaluronic acid or a corticosteroid (e.g., betamethasone, triamcinolone). In one embodiment, the active ingredient comprises a calcium salt. Examples of suitable calcium salts comprise calcium phosphates and calcium sulfates. Examples of calcium phosphates comprise beta-TCP and hydroxyapatite.

In one embodiment, the active ingredient is selected from the group consisting of gentamicin, tobramycin, amikacin, clindamycin, daptomycin, vancomycin, teicoplanin, dalbavancin, fosfomycin, linezolid, eperezolid, colistin, meropenem, fluconazole, micafungin, caspofungin, metronidazole, moxifloxacin, ofloxacin, levofloxacin, ciprofloxacin, rifamycin and rifampicin.

In one embodiment, the medical fluid comprises an aqueous solution of an active ingredient described herein.

The spacer comprises an outer surface. A plurality of delivery valves are arranged on this outer surface. The delivery valves are designed and configured to deliver a medical fluid. The delivery valves are connected to one another via a channel. The channel is arranged in an interior of the spacer. In some embodiments, the spacer comprises an inlet opening. The inlet opening is preferably arranged on the outer surface of the spacer. The inlet opening is designed and configured to introduce medical fluid into the channel. In one embodiment having an inlet opening and a plurality of delivery valves, the channel connects the delivery valves both to one another and to the inlet opening.

The delivery valves are preferably designed and configured to open reversibly depending on the pressure of a fluid within the channel in order to deliver the fluid from the delivery valves. This enables controlled delivery of the fluid from the spacer.

In some embodiments, the delivery valves are arranged substantially uniformly distributed over the entire outer surface of the spacer so that a fluid can be delivered uniformly to the entire surroundings of the spacer. In this case, the fluid can be delivered in all spatial directions.

For the uniform delivery of a medical fluid into the surroundings of the spacer, it may also be advantageous to provide a sufficient amount of delivery valves for this purpose in relation to the surface of the spacer. In one embodiment, the spacer therefore comprises at least one delivery valve per 16.0 cm² of outer surface of the spacer, further preferably at least one delivery valve per 9.0 cm² of outer surface of the spacer.

The delivery valves can form a unit with the material of the spacer, i.e., be integrated into the spacer. Examples of this include the slit valves described herein, wherein, for example, openings on the outer surface of the spacer are coated and covered with a rubber-elastic material. Such a rubber-elastic material suitable for production of slit valves is also referred to herein as “first material.”

The delivery valves can be modular so that the remaining part of the spacer can be manufactured separately from the delivery valves and then the valves can be joined to the remaining part of the spacer. This allows for flexible and cost-effective production, as, for example, similar modular delivery valves can be used in differently shaped spacers.

In some embodiments, the delivery valves each have a sleeve-shaped housing. In some embodiments, the spacer has a plurality of receptacles that enable such delivery valves to be joined to the spacer. In some embodiments, the delivery valves are connectable or connected to the spacer by positive and/or non-positive engagement of the housings into the receptacles.

The delivery valve is preferably a pressure relief valve, i.e., a valve that opens above a defined pressure. This pressure is referred to herein as “limit pressure.” Preferably, all delivery valves of the spacer have the same limit pressure. Furthermore, valves are preferred which are “contactless” with respect to the surroundings of the spacer, i.e., which are designed such that no parts move towards the surrounding tissue when they are opened. This can prevent irritation of the sensitive patient’s tissue, in particular in the presence of inflammation. Examples of such delivery valves are those which have a rubber-elastic membrane with a slit-shaped opening arranged therein, as described below.

In one embodiment, the delivery valves are designed and configured to open above a limit pressure of a fluid inside the channel, which is 1 bar (10^5 Pa) higher than the pressure outside the spacer, and to close fluid-tightly below this limit pressure. This means that the delivery valves are closed when the overpressure of fluid in the channel is less than 1 bar, and the delivery valves are open when the overpressure of fluid in the channel is 1 bar or higher. “Overpressure” refers here to the difference between the atmospheric pressure and the pressure of the fluid in the channel of the spacer.

In one embodiment, the delivery valves each comprise a first material. The first material is rubber-elastic. A rubber-elastic material is characterized by the fact that it automatically returns to its original shape after mechanical deformation. According to the present invention, this property, as explained further below, gives a slit arranged in the resilient material the ability to open or close depending on pressure.

The delivery valves may, for example, comprise a first material which is configured in the form of a coating on the outer surface of the spacer. The channel of the spacer may open into an opening on the outer surface of the spacer, which opening may be provided with such a coating. In this case, the coating can form a sealing boundary for such an opening.

In one embodiment, the delivery valves each have a housing into which such a first material is introduced. For example, the housing may have a substantially cup-shaped geometry having an opening. In such an embodiment, the first material can seal the opening in the housing of the delivery valve in a fluid-tight manner. The first material can be disc-shaped or cup-shaped.

The delivery valves can be connectable to the spacer in a positive, non-positive and/or integral manner. For example, the delivery valves can be connectable or connected to the spacer by pressing and/or gluing and/or welding and/or screwing. Such a connection may exist between the housing of a delivery valve and a receptacle of the spacer described herein.

In one embodiment, the first material has a Shore A hardness in the range of 30 to 80. In one embodiment, the first material has a Shore A hardness of 40 to 70, or 50 to 60, for example approximately 55. The Shore A hardness is determined according to ASTM D2240. Elastomers in this range of Shore A hardness have very good restoring force and are very suitable for the manufacture of a rubber-elastic disc or rubber-elastic coating as described herein as part of a delivery valve. Slits in such elastomers close automatically and quickly when the pressure acting on them drops. In one embodiment, the first material has a Shore A hardness in the range of 75 to 95.

The first material is preferably a polymer, in particular an elastomer. Preferably, the first material comprises or consists of a medically acceptable elastomer. Examples of medically acceptable elastomers comprise silicone elastomers, thermoplastic elastomers (TPEs), polyisoprene, butyl rubber, nitrile rubber, ethylene propylene diene monomer (EPDM), chloroprene rubber, fluoroelastomers, perfluoroelastomers, and polyacrylate elastomers. Examples of silicone elastomers comprise polydimethylsiloxane (PDMS) and liquid silicone rubber (LSR).

Examples of thermoplastic elastomers (TPEs) comprise styrene block copolymers (SBCs) such as styrene-ethylene-butylene-styrene (SEBS), thermoplastic polyurethanes (TPU), and thermoplastic copolyesters (TCE). Examples of polyisoprene comprise natural rubber and synthetic polyisoprene. Examples of butyl rubber comprise bromobutyl rubber (BIIR) and chlorobutyl rubber (CIIR). A preferred polyurethane is a polyether urethane. Polyether urethanes can be produced by polyaddition reaction of a polyetherpolyol with a diisocyanate.

In one embodiment, the first material has a thermoplastic elastomer. In one embodiment, the first material consists of a thermoplastic elastomer. In one embodiment, the first material has a polyether urethane or ethylene propylene diene rubber. In one embodiment, the first material comprises a polyether urethane. In one embodiment, the first material consists of polyether urethane.

In one embodiment, the first material has only a single elastomer, i.e., no second elastomer is mixed with the first material. In one embodiment, the first material has at least two different materials, for example two different elastomers. This allows, for example, the hardness of the first material to be set to a desired target value. The first material may comprise a copolymer. The copolymer may be a thermoplastic elastomer. Preferred are copolymers which comprise a soft segment and a hard segment in their molecular chain. The ratio between the soft segment and the hard segment within the molecular chain of the copolymer can be used to adjust the physical properties of such a copolymer, for example the Shore A hardness of the copolymer. The hard segment can be connected to the soft segment using a connector (linker).

Furthermore, the first material can comprise an additive to adjust the hardness. Examples of such additives are fillers and plasticizers. Examples of fillers comprise silica, titanium dioxide, calcium carbonate, barium sulfate and carbon.

Examples of plasticizers comprise adipates, trimellitates, citrate-based plasticizers, and esters of polyhydric alcohols.

For example, TOTM (tris(2-ethylhexyl)trimellitate), DINCH (diisononylcyclohexane-1,2-dicarboxylate), ATBC (acetyltributylcitrate), or DEHA (di(2-ethylhexyl)adipate) can be used as plasticizers.

In one embodiment, the first material is free of plasticizers. In one embodiment, the first material is free of fillers. In one embodiment, the first material is free of endocrine disrupting substances such as phthalates or bisphenols.

In one embodiment, the first material may further comprise a lubricant. Preferably, the lubricant is medically acceptable. Preferably, the lubricant is free of polyhalogenated substances and silicones. In one embodiment, the lubricant has a natural product, for example a lipid, a triglyceride or a biopolymer.

In one embodiment, the first material may have a Young’s modulus of elasticity of 1.2 × 10^7 Pa to 2.1 x × 10^7 Pa, for example 1.3 × 10^7 Pa to 2.0 x × 10^7 Pa, 1.4 × 10^7 Pa to 1.9 x × 10^7 Pa, 1.5 × 10^7 Pa to 1.8 x × 10^7 Pa, or 1.5 × 10^7 Pa to 1.7 x × 10^7 Pa. In one embodiment, the first material may have a Young’s modulus of elasticity of approximately 1.6 × 10^7 Pa. In one embodiment, the first material may have a Young’s modulus of elasticity of 2000 to 2500 psi. The latter corresponds approximately to 1.4 × 10^7 Pa to 1.7 × 10^7 Pa.

The Young’s modulus of elasticity can be determined according to ASTM D412. The first material is preferably sterilizable using common sterilization processes, i.e., resistant to UV radiation, gamma radiation and treatment with ethylene oxide within the scope of these processes. The first material is preferably moldable using standard extrusion and/or injection molding processes.

The first material may comprise a slit. In one embodiment, the slits have a straight, curved, star-shaped, cross-shaped or horseshoe-shaped shape. In one embodiment, the slits each have a total length in the range of 0.4 mm to 3.0 mm, for example in the range of 0.8 mm to 2.5 mm, or in the range of 1.0 to 2.0 mm.

In one embodiment, each of the delivery valves comprises a first material having a respective slit. In one embodiment, each of the delivery valves has exactly one, i.e., no more than one, slit in the first material.

In one embodiment, all the slits of the delivery valves are each of substantially the same length. In one embodiment, all the slits are of a length that deviates by no more than 25% up or down from a certain value, for example a length of 1.00 mm +/- 25%, i.e., e.g., 0.75 mm to 1.25 mm.

In one embodiment, the slits are configured to reversibly open and close by means of a resilient restoring force of the first material. In this case, the slits can be opened by an overpressure of a fluid in the channel, in that the first material is pushed apart by the fluid when a predetermined limit pressure of the fluid is exceeded.

In one embodiment, the slits can be or are produced by sectionally separating the first material, without removing parts of the first material in the process. In this case, the slits can be made, for example, by punching with a blade. Such slits are completely closed unless there is a pressure difference across the two opposite sides of the first material. This prevents tissue or liquid from penetrating into the spacer from the outside.

In one embodiment, the slits have walls that touch one another when a delivery valve is in the closed state and move away from one another when a delivery valve is opened.

In a further embodiment, the channel has an inlet opening for receiving a fluid, wherein the inlet opening is preferably arranged on the outer surface of the spacer.

Furthermore, the spacer can have a fluid connector. The fluid connector can be used to connect a vessel or a fluid-conducting connection to receive a liquid into the spacer. An example of a fluid connector is a syringe connection, e.g., a Luer-Lock connector. Using such a connector, a commercially available Luer-Lock syringe can be connected to the inlet opening of the spacer in a liquid-tight and fluid-conducting manner. The fluid connector can be detachably connectable to the inlet opening. The fluid connector can be operatively connectable to the spacer.

The spacer may further comprise a supply line that is connectable or connected to the inlet opening. The supply line may preferably be operatively connectable or connected to the inlet opening. In one embodiment, the spacer has a fluid connector that is connectable or connected to the inlet opening via a supply line. The supply line can, for example, have a tube made of a medically acceptable material.

In one embodiment, the spacer may comprise a check valve. The check valve is preferably designed to prevent fluid from escaping from the spacer through the inlet opening. The check valve may be operatively connectable or connected to the inlet opening. The check valve may be located directly at the inlet opening or may be arranged on the supply line or fluid connector. Thus, fluid can be introduced into the spacer through the inlet opening without the fluid flowing back through the inlet opening.

For a secure connection of the supply line to the inlet opening, the spacer may further comprise a locking mechanism, which is preferably arranged at the inlet opening. In one embodiment, the locking mechanism comprises, for example, one or more movable locking elements which are designed and configured to move radially inward when the supply line is inserted into the inlet opening and engage in corresponding grooves or recesses on the inlet opening. The locking mechanism may preferably be spring-loaded to allow for engagement of the locking elements at the inlet opening.

The locking mechanism may have a release which is designed and configured to detach the locking elements.

In a further embodiment, the spacer further comprises a trocar. A trocar comprises a pointed end that allows it to penetrate tissue. Furthermore, a trocar comprises a shaft along which the trocar can be guided. The shaft may comprise a cylindrical cavity, similar to a cannula.

The trocar is preferably connectable to the supply line described herein. Preferably, the trocar can be detachably connected to a distal end of the supply line. Using the trocar, a medical user can insert the supply line into patient’s tissue and thereby position and fix it at a desired location. A trocar can allow for gentle and precise penetration of a target tissue. In this case, a channel can be created to the desired administration site, allowing the spacer for administering a medical fluid to be positioned accordingly. In this case, the supply line can be introduced into the patient’s tissue through the channel created with the help of the trocar. Preferably, the trocar is removable to allow a medical fluid to be received through the supply line following positioning of the supply line using the trocar. In this case, the trocar can, for example, be removed from the supply line and replaced with a fluid connector, such as a Luer-Lock connector. A medical fluid can then be added to the supply line using a Luer-Lock syringe, for example, in order to deliver it to a target location on the patient’s tissue using the device.

In one embodiment, the inlet opening described herein may be configured in an identical manner to the receptacles described herein. In one embodiment, the inlet opening is configured differently from the receptacles described herein.

The spacer can be adapted for different locations. In one embodiment, the spacer is selected from the group consisting of a knee joint spacer, a hip joint spacer, a vertebral body spacer and an intramedullary nail spacer.

The knee joint spacer can be a one-piece or multi-piece knee joint spacer. The hip joint spacer can be a one-piece or multi-piece hip joint spacer. The vertebral body spacer can be a one-piece or two-piece vertebral body spacer. In the multi-piece spacers, the delivery systems described herein can be present either in just one of the two parts or in both parts.

The spacers described here, for example the hip joint spacers or the knee joint spacers, can each have a first sub-element and a second sub-element. The first sub-element and/or the second sub-element can be intended for introduction into a patient’s bone. In this case, anchoring can be done with bone cement.

In one embodiment, both the first sub-element and the second sub-element comprise delivery valves.

In one embodiment, the spacer further comprises a cementing region which has no inlet openings, delivery valves, or inlet openings. This cementing region is preferably designed and configured to be fixed in the bone of a patient to be treated by using a bone cement. In this way, the spacer can be held in position at a desired target location after implantation.

Furthermore, it is possible to design the present invention described herein as an implant intended to remain permanently in a patient. For example, an osteosynthesis plate as described herein may be provided with a channel and the delivery valves described herein. Accordingly, in one aspect, the present invention also provides an implant for delivering a medical fluid to a patient, wherein the implant has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves. The implant can be designed as a joint implant, for example as a hip joint implant, shoulder joint implant, or knee joint implant.

In a further embodiment, the spacer or implant described herein is designed and configured to deliver a substantially identical amount of fluid from each of the delivery valves simultaneously. For example, a substantially identical amount may be an amount that has a statistical standard deviation of less than 10% of the arithmetic mean.

In a further embodiment, the spacer or implant described herein is designed and configured to open and/or close all delivery valves simultaneously. Depending on the pressure of a fluid in the channel, the delivery valves are accordingly each either opened or closed, as described herein.

In one embodiment, the delivery valves are each arranged such that they are flush with the outer surface of the spacer. Such a design can prevent the delivery valves from forming projections with respect to the spacer, which could lead to injury or irritation of patient’s tissue.

In one embodiment of the present invention, the channel has a plurality of branches. In this case, each branch preferably has a delivery valve or an inlet opening according to the embodiments described herein. This means that in the case of a branch, a delivery valve or an inlet opening is present on each side arm of the channel, wherein the delivery valves or inlet openings are in this case preferably arranged on the outer surface of the spacer.

In one embodiment, the delivery valves are designed and configured to allow liquid to pass through the delivery valves only unidirectionally. This means that the delivery valves in this case are designed as one-way valves. According to the present invention, this means that no liquid can enter the channel from the outside through a delivery valve as long as the pressure of a fluid in the channel is equal to or higher than the ambient pressure on the outside of the spacer.

In one embodiment, the spacer or implant has a joint element. The joint element may have a joint body. The joint element can movably connect the first sub-element to the second sub-element. The channel can extend from the first sub-element through the joint element into the second sub-element. In this case, the channel can be arranged along a central axis of the joint element.

In one embodiment, the channel defines a connection axis along its extension direction from the first sub-element through the joint element. This axis is defined in a state in which no external force acts on the spacer. The joint element can be designed and configured to allow a movement of the second sub-element by at least 20° at an angle to this connection axis. In one embodiment, this angle is at least 30° or at least 40°. For example, the second sub-element can be laterally bendable relative to the first sub-element by at least 20°, at least 30° or at least 40°. In order to allow such movement, the joint body may comprise a rubber-elastic second material. In one embodiment, the joint body consists of the second material. The second material may be identical to the first material described herein, or the second material may be different from the first material described herein. The second material can have a Shore A hardness in the range of 15 to 80. In one embodiment, the second material has a Shore A hardness in the range of 20 to 70 or in the range of 30 to 60. The second material may have a closed porosity.

The joint body may have an outer diameter which is between 0.25 times and 0.75 times the outer diameter of the surrounding first sub-element or second sub-element. These two outer diameters are preferably defined in a direction which is orthogonal to the connection axis described above. Preferably, these two outer diameters are each defined along the same line.

In one embodiment, the channel has a diameter having a ratio to an outer diameter of the joint body which is in a range of 1:1 to 1:6. In this case, the diameter of the channel is defined as the clear width of the channel.

In one embodiment, the spacer has an anchoring which connects the joint body to the first sub-element and/or to the second sub-element. In one embodiment, the anchoring connects the joint body directly to the first sub-element or to the second sub-element. In one embodiment, the anchoring connects the joint body to the first sub-element. In one embodiment, the anchoring connects the joint body to the second sub-element. In one embodiment, a first anchoring connects a first joint body to the first sub-element, and a second anchoring connects a second joint body to the second sub-element.

In one embodiment, the channel extends through the anchoring. In one embodiment, the channel extends through the joint body. In one embodiment, the channel extends through the anchoring and through the joint body. In one embodiment, the anchoring has a substantially cylindrical shape. The anchoring can be designed as a screw or bolt, for example. Accordingly, the anchoring can include a thread. The anchoring may also include peripheral grooves, projections or other locking means. The anchoring may include a cavity to form part of the channel as described herein.

The anchoring can be connected to the second material in a positive, non-positive and/or integral manner. The anchoring can be connected to the first sub-element in a positive, non-positive and/or integral manner. The anchoring can be connected to the second sub-element in a positive, non-positive and/or integral manner.

In one embodiment, the joint element, preferably the joint body of the joint element, may have a delivery valve as described herein. In this case, the delivery valve can be arranged in the second material. The second material may comprise a slit. In one embodiment, this slit is configured to reversibly open and close by means of a resilient restoring force of the second material.

In one embodiment, the spacer comprises a plurality of joint elements. The joint elements can be connected to one another directly or indirectly. For example, a plurality of joint bodies can be directly connected to one another. For this purpose, joint bodies can have, for example, threads or locking elements, such as recesses and/or projections. The joint bodies may be connected to one another by intermediate anchorings as described herein.

In addition to the first sub-element and the second sub-element, the spacer may comprise further such sub-elements, each of which is connected to one another by the joint elements described herein. This allows for greater mobility of the spacer. For example, in such an embodiment, the spacer can be curved in different directions simultaneously, in a similar way to how the vertebral joints in the spine allow this.

A further aspect of the present invention relates to a kit for producing a spacer described herein, which has a plurality of sub-elements described herein and one or more joint elements described herein. In one embodiment, the kit comprises a plurality of interchangeable sub-elements. These sub-elements can be of different sizes and/or geometries, for example different lengths and/or thicknesses. This allows a medical user to individually adapt the spacer to a patient to be treated.

In one embodiment, the kit is designed and configured for producing an implantable spacer for delivering a medical fluid to a patient, wherein the spacer has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves.

A second aspect of the present invention relates to a medical fluid for use in a medical procedure, wherein the procedure comprises the following steps:

providing a spacer described herein;

introducing a medical fluid into the channel of the spacer;

pressurizing the medical fluid in the channel to open the delivery valves in a pressure-dependent manner and deliver the fluid to a patient.

The medical procedure preferably includes the treatment of inflammation, mechanical injury (trauma), infection or oncological disease. The procedure preferably comprises the treatment of a diseased bone or joint. The infection can be, for example, osteomyelitis or osteitis. Furthermore, the procedure may include the treatment of pain.

In one embodiment, the medical procedure involves a joint infection, for example a hip joint infection, knee joint infections, shoulder joint infections, or spondylodiscitis. In one embodiment, the treated joint infection is surgically caused, for example due to a surgical procedure to implant an artificial joint. In some embodiments, a joint infection is treated using a medical fluid comprising an antibiotic and/or an antifungal agent.

A further embodiment according to the second aspect of the present invention relates to a medical fluid for use in a medical procedure as described above, wherein the fluid comprises an active ingredient. In principle, all of the active ingredients described herein can be used. In one embodiment, the fluid comprises an active ingredient selected from the group consisting of an antibiotic, an antiphlogistic agent, an anesthetic agent, and a cytostatic agent.

In one embodiment, the active ingredient is selected from the group consisting of gentamicin, tobramycin, amikacin, clindamycin, daptomycin, vancomycin, teicoplanin, dalbavancin, fosfomycin, linezolid, eperezolid, colistin, meropenem, fluconazole, micafungin, caspofungin, metronidazole, moxifloxacin, ofloxacin, levofloxacin, ciprofloxacin, rifamycin and rifampicin.

In one embodiment, the medical fluid is an aqueous solution of an active ingredient described herein.

In one embodiment, the medical procedure comprises implanting the spacer into a patient’s tissue, preferably in the region of a joint or at the site of a fracture. In one embodiment, the medical procedure comprises penetrating patient’s tissue using a trocar to deliver a supply line of the spacer from inside a patient’s body through the skin to the outside.

In one embodiment, the medical fluid is introduced into the channel of the spacer using a pump, for example using a syringe pump. In one embodiment, the medical fluid is delivered to the patient continuously over a period of several minutes, several hours, or several days. In one embodiment, the medical fluid is repeatedly delivered to the patient. For example, the medical fluid can be delivered to the patient once a day or several times a day, for example twice, three times, or more than three times a day. In one embodiment, the medical fluid is delivered depending on a health parameter of the patient. The health parameter can be, for example, a diagnostic measurement or the patient’s pain level.

In one embodiment, the medical fluid is dosed using a manual or automatic control.

A further aspect of the present invention relates to a medical treatment procedure, the procedure comprising the following steps:

providing a spacer, implant or kit described herein,

introducing a medical fluid into the channel of the spacer or implant,

pressurizing the medical fluid in the channel to open the delivery valves in a pressure-dependent manner and deliver the fluid to a patient.

The above embodiments regarding a medical fluid for use in a medical procedure apply accordingly.

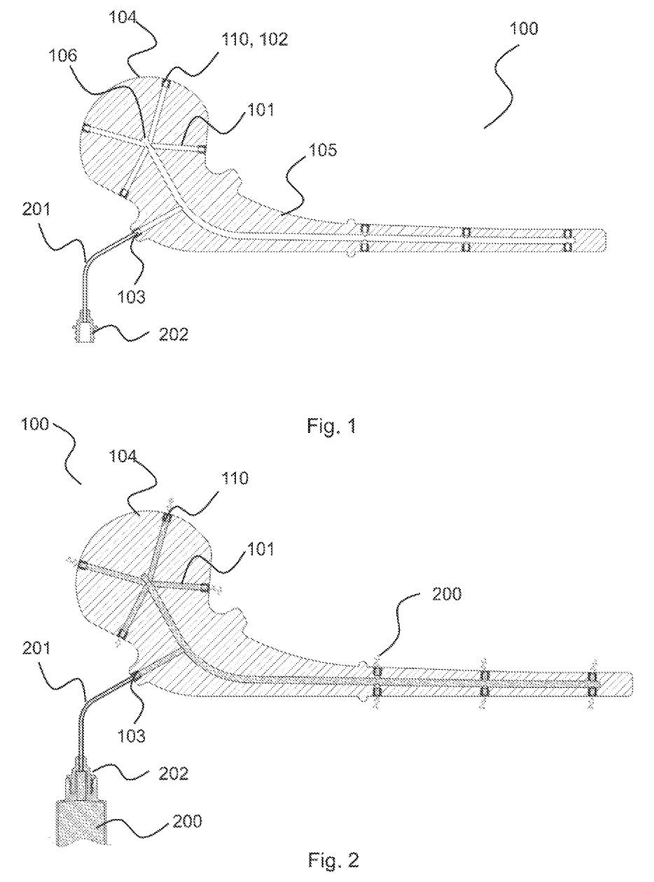

FIG. 1 is a cross section of a first embodiment of a spacer 100 according to the present invention. Within the spacer a fluid-conducting channel 101 is arranged. The channel 101 has branches 106. The spacer has an outer surface 104 on which receptacles 102 are arranged. Delivery valves 110 are introduced into the receptacles 102. In the example shown here, one of the receptacles 102 is designed as an inlet opening 103 in order to provide a fluid-conducting connection to a supply line 201. In this case, a proximal end of the supply line 201 engages positively or non-positively into the inlet opening 103. The supply line 201 has a distal end which is fluidically connected to a fluid connector 202. The fluid connector 202 is designed here as a Luer-Lock connector. The delivery valves 110 are located at different positions on the outer surface 104 of the spacer in order to enable a delivery of a medical fluid into the spacer’s surroundings that is as uniform as possible. The channel 101 connects the inlet opening 103 with the delivery valves 110, so that a fluid received through the inlet opening 103 can arrive, via the channel 101, at the delivery valves 110. The spacer also has a cementing region 105 which has no inlet openings, delivery valves or inlet openings. This cementing region 105 is designed and configured to be fixed in the bone of a patient to be treated, using a bone cement. In this way, the spacer can be held in position at a desired target location after implantation.

FIG. 2 shows a cross section of a spacer according to FIG. 1, which is filled with a medical fluid 200. The design of the spacer shown here allows a medical fluid to be filled, using a syringe, via a fluid connector 202, a supply line 201 and inlet opening 103, into the channel 101. Due to pressurization of the fluid 200 within channel 101, the delivery valves 110 open to remove the fluid from the spacer 100 to a patient’s target tissue. The delivery valves 110 are designed and configured to open above a predetermined pressure of the fluid 200 in the channel 101. This makes it possible to achieve a temporally and quantitatively uniform delivery of the fluid 200 via all the delivery valves 110.

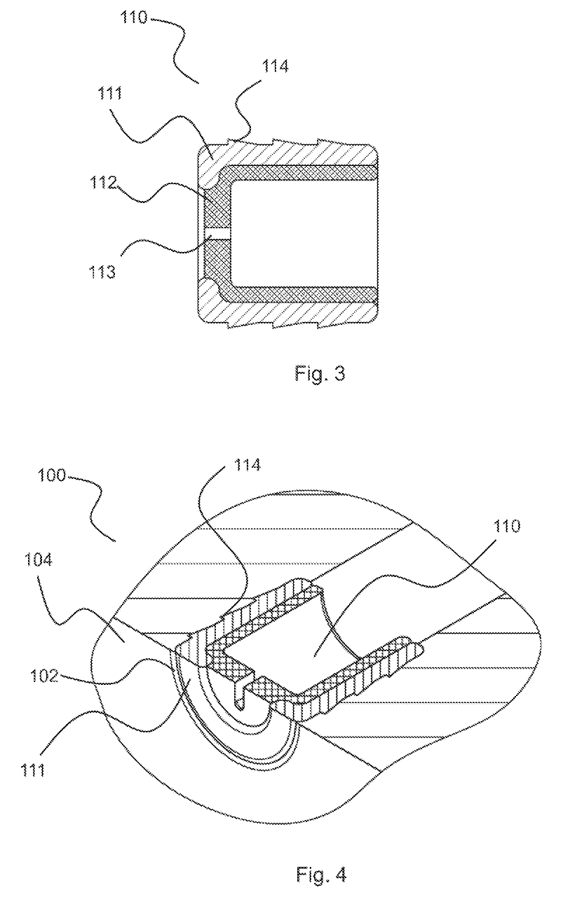

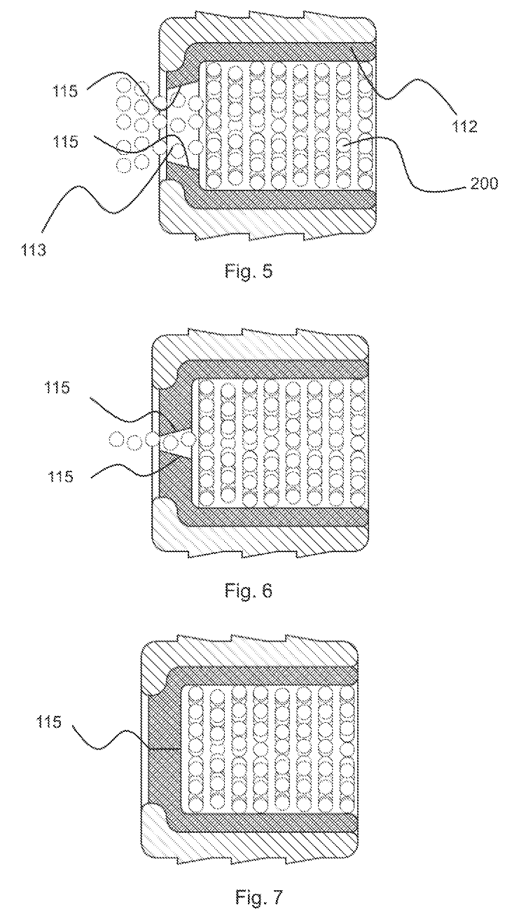

FIG. 3 is a cross-sectional view of an embodiment of delivery valve 110. This embodiment of delivery valve 110 is constructed in a modular design so that it can be manufactured separately and introduced into spacer 100 according to the present invention. The delivery valve 110 has a housing 111, the housing 111 comprising projections 114 which are designed for a firm connection with a receptacle of the spacer. In an interior of the delivery valve, a rubber-elastic first material 112, comprising a slit 113, is arranged. The slit 113 is designed and configured to open or close in a pressure-dependent manner, as shown in more detail in the following FIG. 5 to 7. The slit 113 is shown here in a partially opened state.

FIG. 4 shows a section of an embodiment of spacer 100 according to the present invention, modular delivery valve 110 engaging non-positively into receptacle 102 of the spacer. In this case, the delivery valve 110 is arranged such that the housing 111 of the delivery valve 110 is flush with the outer surface 104 of the spacer. This prevents the arrangement of the delivery valve 110 in the receptacle 102 forming projections which could pose a risk of injury to patients. The delivery valve 110 is shown here in a partially opened state.

FIG. 5 shows an embodiment of delivery valve 110 in a fully open state. Due to increased pressure of the fluid 200 which flows from a channel of the spacer into the delivery valve 110, the slit 113 opens, which is arranged in rubber-elastic first material 112. The slit 113 has slit walls that separate and move away from one another due to the increased pressure of the fluid 200, so that the slit 113 opens and allows delivery of the fluid 200 from the delivery valve 110.

FIG. 6 shows an embodiment of delivery valve 110 in a partially open state. In the drawing shown here, the pressure of the fluid 200 is only slightly above the limit pressure at which the slit 113 opens. The two slit walls 115 are located close to one another and allow, compared to the state shown in FIG. 5, only a relatively small delivery quantity and delivery speed of the fluid 200 from the delivery valve 110.

FIG. 7 shows an embodiment of delivery valve 110 in a closed state. In the drawing shown here, the pressure of the fluid 200 is below the limit pressure at which slit 113 would open. The two slit walls 115 touch one another and lie completely against one another, so that the slit 113 is closed and does not allow any flow of fluid 200.

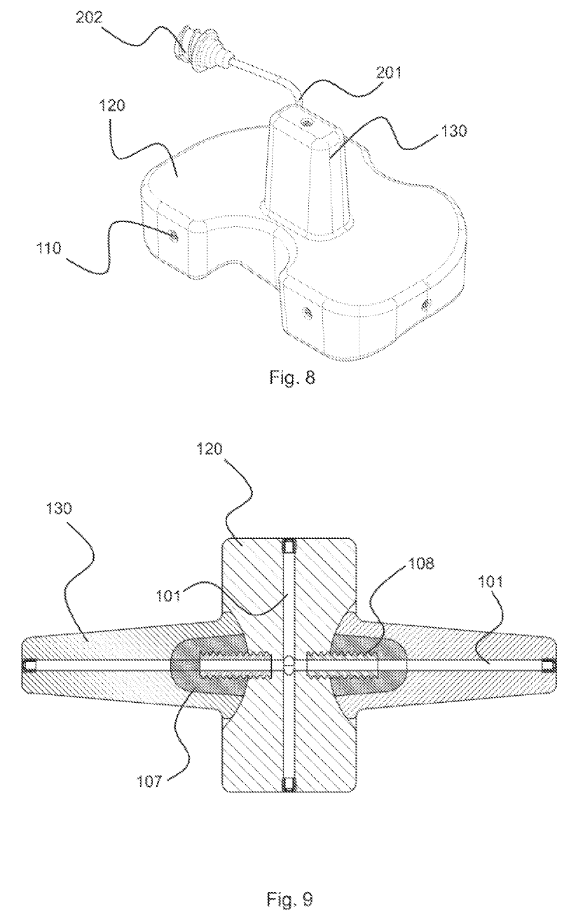

FIG. 8 shows an embodiment of a spacer according to the present invention, which in the example shown here is designed as a knee joint spacer. The spacer has a first sub-element 120 and a second sub-element 130, both the first sub-element 120 as well as the second sub-element 130 each comprising delivery valves 110 which are designed and configured to deliver and release a medical fluid. The spacer also has supply line 201 with fluid connector 202 for introducing a medical fluid as previously described herein.

FIG. 9 shows a further embodiment of a spacer according to the present invention in a cross-sectional view, which is designed as a knee joint spacer. This embodiment has plurality of delivery valves 110 which are connected to one another and to inlet opening 103 via channel 101. Furthermore, in the embodiment shown here the spacer comprises a joint element 107, which comprises a rubber-elastic material. The joint element 107 is positively connected to second sub-element 130 of the spacer. Furthermore, the joint element 107 is positively and/or non-positively connected to first sub-element 120 of the spacer via an anchoring 108. The channel 101 extends within the spacer through the first sub-element 120, the anchoring 108, the joint element 107 and the second sub-element 130. The joint element 107 allows a lateral rotation of the second sub-element 130 with respect to the first sub-element 120. The embodiment shown here has two similar second sub-elements 130 which, as described above, are each connected to the same first sub-element 120 via anchoring 108 and joint element 107.

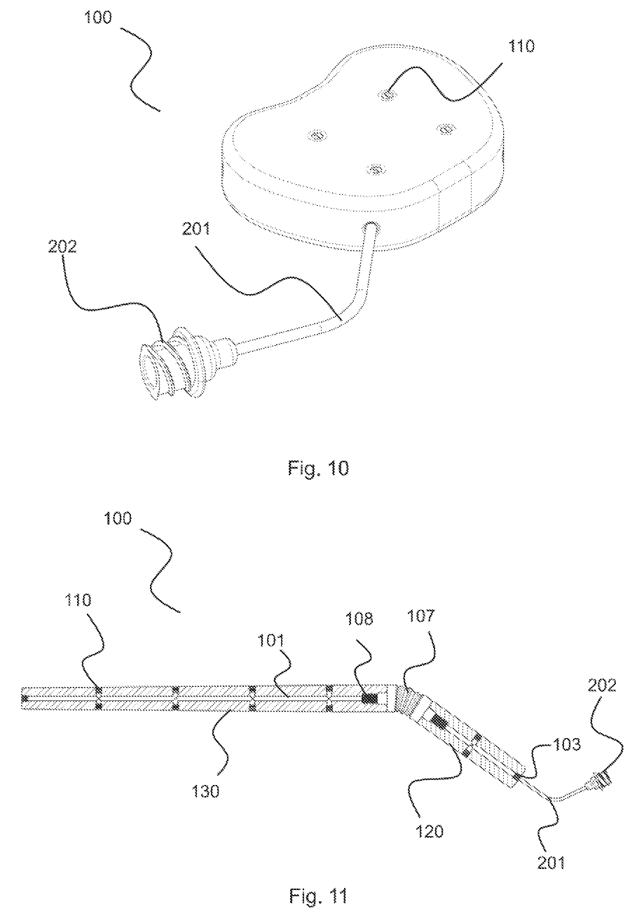

FIG. 10 shows a further embodiment of a spacer according to the present invention, which is designed as a vertebral body spacer. The spacer 100 has plurality of delivery valves 110, supply line 201 and fluid connector 202. The supply line 201 is, as described herein, fluidically connected via channel 101 (not shown here) to the delivery valves 110.

FIG. 11 shows a further embodiment of a spacer according to the present invention, which is designed as an intramedullary nail spacer. The spacer 100 has first sub-element 120 and second sub-element 130, the first sub-element 120 being connected via joint element 107 to the second sub-element 130. The joint element 107 can be connected, similarly to FIG. 9, using anchoring 108, to the first sub-element 120 and the second sub-element 130. The first sub-element 120 has inlet opening 103 with supply line 201 and fluid connector 202, the inlet opening 103 being fluidically connected via channel 101 to plurality of delivery valves 110, the delivery valves 110 being arranged both on the first sub-element 120 and on the second sub-element 130. The delivery valves 110 are uniformly distributed over the outer surface of the spacer, i.e., over the outer surfaces of the first sub-element 120 and the second sub-element 130, in order to ensure the most uniform possible delivery of a medical fluid to the entire surroundings of the spacer 100.

### LIST OF REFERENCE SIGNS

100 Spacer

101 Channel

102 Receptacle

103 Inlet opening

104 Outer surface

105 Cementing region

106 Branch

107 Joint element

108 Anchoring

110 Delivery valve

111 Housing

112 First material

113 Slit

114 Projection

115 Slit wall

120 First sub-element

130 Second sub-element

200 Fluid

201 Supply line

202 Fluid connector

What is claimed is:

1. An implantable spacer for delivering a medical fluid to a patient, wherein the spacer has an outer surface and a plurality of delivery valves arranged on the outer surface, wherein the delivery valves are each fluidically connected to one another by a channel arranged within the implant, and wherein the delivery valves are designed and configured to open reversibly depending on the pressure of a fluid within the channel in order to deliver the fluid from the delivery valves.

2. The implantable spacer according to claim 1, wherein the delivery valves each have a sleeve-shaped housing, and wherein the spacer has a plurality of receptacles, wherein the delivery valves can be connected to the spacer by positive and/or non-positive engagement of the housings into the receptacles.

3. The implantable spacer according to claim 1, wherein the delivery valves each comprise a first material, wherein the first material is rubber-elastic, and the first material further comprises a slit in each case.

4. The implantable spacer according to claim 3, wherein the slits are designed to reversibly open and close by means of an elastic restoring force of the first material of the delivery valve.

5. The implantable spacer according to claim 3, wherein the first material has a Shore A hardness of 30 to 80, preferably 40 to 70, more preferably 50 to 60.

6. The implantable spacer according to claim 1, wherein the channel has an inlet opening for receiving a fluid, wherein the inlet opening is preferably arranged on the outer surface of the spacer.

7. The implantable spacer according to claim 2, wherein the inlet opening is designed in an identical manner to the receptacles, or wherein the inlet opening is designed in a manner different from the receptacles.

8. The implantable spacer according to claim 1, wherein the spacer is selected from the group consisting of a knee joint spacer, a hip joint spacer, a vertebral body spacer and an intramedullary nail spacer.

9. The implantable spacer according to claim 1, wherein the spacer is designed and configured to deliver a substantially identical amount of fluid from each of the delivery valves simultaneously.

10. The implantable spacer according to claim 1, wherein the spacer is designed and configured to open and/or close all delivery valves simultaneously.

11. The implantable spacer according to claim 1, wherein the delivery valves are each arranged such that they are flush with the outer surface of the spacer.

12. The implantable spacer according to claim 1, wherein the channel has a plurality of branches, and wherein preferably each branch has a delivery valve or an inlet opening which is preferably arranged on the outer surface of the spacer.

13. The implantable spacer according to claim 1, wherein the delivery valves are designed and configured to allow liquid to pass only unidirectionally through the delivery valves.

14. A medical procedure, comprising the following steps:
providing an implantable spacer according to claim 1,
introducing a medical fluid into the channel of the spacer,
pressurizing the medical fluid in the channel in order to open the delivery valves in a pressure-dependent manner and deliver the fluid to a patient.

15. The medical fluid for use in the method according to claim 14, wherein the fluid comprises an active ingredient selected from the group consisting of an antibiotic, an antiphlogistic agent, an anesthetic agent and a cytostatic agent.

16. The implantable spacer according to claim 2, wherein the delivery valves each comprise a first material, wherein the first material is rubber-elastic, and the first material further comprises a slit in each case.

17. The implantable spacer according to claim 4, wherein the first material has a Shore A hardness of 30 to 80, preferably 40 to 70, more preferably 50 to 60.
